(19) United States
(12) Patent Application Publication
(10) Pub. No.: US 2014/0236218 A1
(43) Pub. Date: Aug. 21, 2014

(54) FRAME-BASED VENA CAVA FILTER

(71) Applicant: Cook Medical Technologies LLC, Bloomington, IN (US)

(72) Inventors: Alexandra Elizabeth Jantzen, Durham, NC (US); Grant T. Hoffman, Bloomington, IN (US)

(73) Assignee: Cook Medical Technologies LLC, Bloomington, IN (US)

(21) Appl. No.: 14/259,836

(22) Filed: Apr. 23, 2014

Related U.S. Application Data
(62) Division of application No. 13/186,627, filed on Jul. 20, 2011.
(60) Provisional application No. 61/365,999, filed on Jul. 20, 2010.

(51) Int. Cl. A61F 2/01

(52) U.S. Cl. CPC A61F 2/01; A61F 2002/016; A61F 2002/011; A61F 2002/018
USPC 606/200

(57) ABSTRACT

A filter device for capturing thrombi in a body vessel of a patient is disclosed. The device comprises first and second portions, each comprising a wire forming a zig-zag configuration. The configuration comprises a series of straight sections and a plurality of bends connected to the straight sections. The straight sections are joined by the bends, defining a first cell size and a second cell size. The filter device further comprises a filter portion connected to the first and second portions. The filter portion comprises the wire forming a zig-zag configuration having a series of straight sections and a plurality of bends connected to the straight sections. The straight sections of the filter portion are joined by the bends of the filter portion, defining a filter cell size. The filter portion is disposed at the arc of the iliac veins at opening of the inferior vena cava.

### CROSS-REFERENCE TO RELATED APPLICATIONS

This application claims the benefit and is a divisional of U.S. application Ser. No. 13/186,627, filed on Jul. 20, 2011, entitled “FRAME-BASED VENA CAVA FILTER.” This application also claims the benefit of U.S. Provisional Application No. 61/365,999, filed on Jul. 20, 2010, entitled “FRAME-BASED VENA CAVA FILTER.” The entire contents of both are incorporated herein by reference.

### TECHNICAL FIELD

The present invention relates to medical devices. More particularly, the invention relates to a vena cava filter that can be percutaneously placed on the arch of the iliac veins at the opening of the inferior vena cava of a patient.

### BACKGROUND

Filtering devices that are percutaneously placed in the vena cava have been available for over thirty years. A need for filtering devices arises in trauma patients, orthopedic surgery patients, neurosurgery patients, or in patients having medical conditions requiring bed rest or non-movement. During such medical conditions, the need for filtering devices arises due to the likelihood of thrombosis in the peripheral vasculature of patients wherein thrombi break away from the vessel wall, risking downstream embolism or embolization. For example, depending on the size, such thrombi pose a serious risk of pulmonary embolism wherein blood clots migrate from the peripheral vasculature through the heart and into the lungs.

A filtering device can be deployed in the vena cava of a patient when, for example, anticoagulant therapy is contraindicated or has failed. In more recent years, filters have been used or considered in preoperative patients and in patients predisposed to thrombosis which places the patient at risk for pulmonary embolism.

The benefits of a vena cava filter have been well established, but improvements may be made. Many filters become off-centered or tilted with respect to the hub of the filter and the longitudinal axis of the body vessel in which it has been inserted. As a result, the filter may potentially become endothelialized therein. In many situations, this is not desired.

### BRIEF SUMMARY

The present invention provides a medical device that filters thrombi in a body vessel and avoids tilting that undesirably results in incomplete or asymmetric coverage and migration.

The present invention generally provides a filter device for capturing thrombi in a body vessel of a patient. In one embodiment, the filter device comprises a first portion comprising a wire formed into a first zig-zag configuration. The first configuration comprises a series of straight sections and a plurality of bends connected to the straight sections. The straight sections are joined by the bends, defining a first cell size of the first portion.

The device further comprises a second portion comprising the wire formed into a second zig-zag configuration. The second configuration comprises a series of straight sections and a plurality of bends connected to the straight sections. The straight sections being joined by the bends defining a second cell size of the second portion.

The device further comprises a filter portion connected to the first and second portions. The filter portion comprises the wire formed into a third closed zig-zag configuration. The third configuration comprises a series of straight sections and a plurality of bends connected to the straight sections. The straight sections are joined by the bends defining a filter cell size of the filter portion. The filter portion is configured to be disposed at the arc of the iliac veins at the opening of the inferior vena cava. The first and second portions are configured to be opposingly disposed in respective left and right iliac veins of the patient.

In this embodiment the device is depressible into a smaller first shape. The straight sections are arranged side by side and closely adjacent one another for insertion into the body vessel and the bends store stress therein in the first shape. The device is expandable into a second shape by the release of the stress stored in the bends. The straight sections of the first and second portions press against the walls of the iliac veins to maintain the walls open while the filter portion filters thrombi therefrom.

In another embodiment, the present invention provides a delivery assembly for placing a filter device for capturing thrombi in a body vessel of a patient. The assembly comprises the filter device and an outer sheath having a body extending from a proximal part to a distal part. The body is tubular and forms a sheath lumen extending there through.

The assembly further comprises an inner member extending from a proximal portion to a distal portion. The inner member is disposed within the sheath lumen and is configured for axial movement relative to the outer sheath. The filter device is co-axially disposed within the sheath lumen and disposed adjacent the distal portion of the inner member. The filter device is deployable through the distal part of the outer sheath by means of the relative axial movement of the inner member.

### BRIEF DESCRIPTION OF THE DRAWINGS

FIG. 1 is a side view of a filter device in an expanded state for capturing thrombi in a body vessel in accordance with one embodiment of the present invention.

FIG. 2 is a side view of the filter device in a collapsed state.

FIG. 3 is an environmental view of the filter device in FIG. 1.

FIG. 4a is a side view of a filter device for capturing thrombi in a body vessel in accordance with yet another embodiment of the present invention.

FIG. 4b is an environmental view of the filter device in FIG. 4a.

FIG. 5a is an assembly for use with one of the filter devices for capturing thrombi in a body vessel.

FIG. 5b is an exploded view of the assembly in FIG. 5a.

### DETAILED DESCRIPTION

The present invention generally provides a medical device that filters thrombi in a body vessel and avoids tilting that undesirably results in incomplete or asymmetric coverage and migration. Embodiments of the present invention include a filter device for capturing thrombi in a body vessel of a patient.

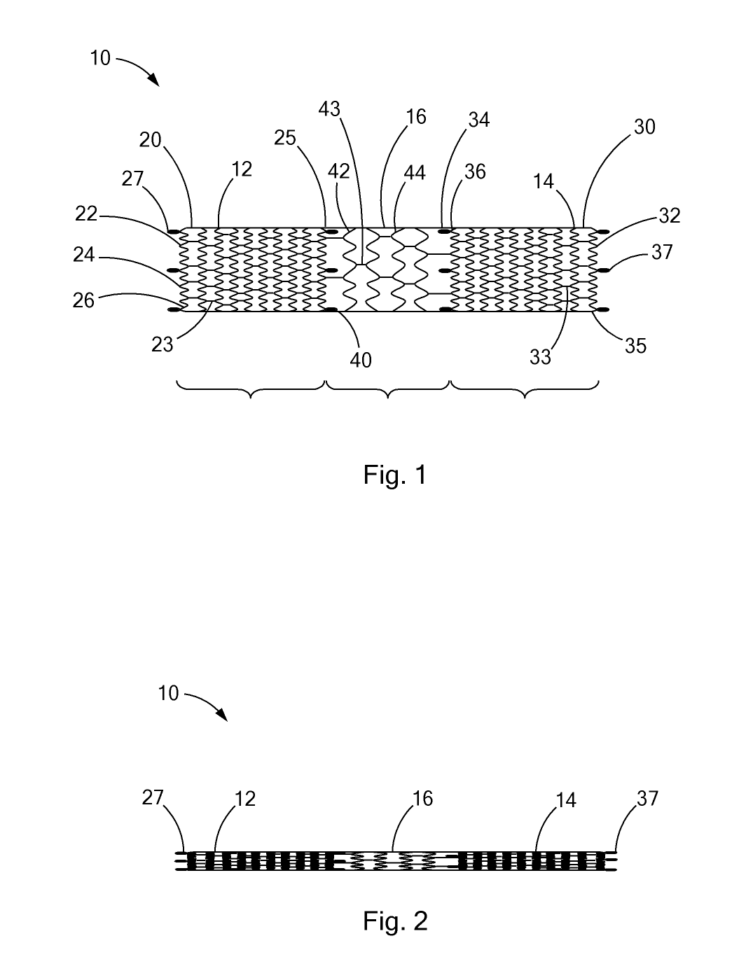

FIGS. 1 and 2 illustrate a filter device for capturing thrombi in a body vessel of a patient in accordance with one embodiment of the present invention. As shown, in FIG. 1, the device 10 comprises a first portion 12 comprising a wire 20 formed into a first zig-zag configuration 22. The first configuration 22 comprises a series of straight sections 23 and a plurality of bends 26 connected to the straight sections 23. In this embodiment, the straight sections 23 are joined by the bends 26 to define a first cell size of first portion 12. As shown, the first portion comprises a first end 24 and a second end 25 extending from the first end 24. Preferably, one of the first end 24 and second end 25 has a marker 27 disposed thereon. As shown in FIGS. 1 and 2, the filter device further comprises a second portion 14 comprising a wire 30 formed into a second zig-zag configuration 32. In this embodiment, the second configuration 32 comprises a series of straight sections 33 and a plurality of bends 36 connected to the straight sections 33. Preferably, the straight sections 33 are joined by the bends 36 to define a second cell size of the second portion 14. In this embodiment, the first cell size is substantially the same as the second cell size. As shown, the second portion comprises a first end 34 and a second end 35 extending from the first end 34. Preferably, one of the first end 34 and second end 35 has a marker 37 disposed thereon.

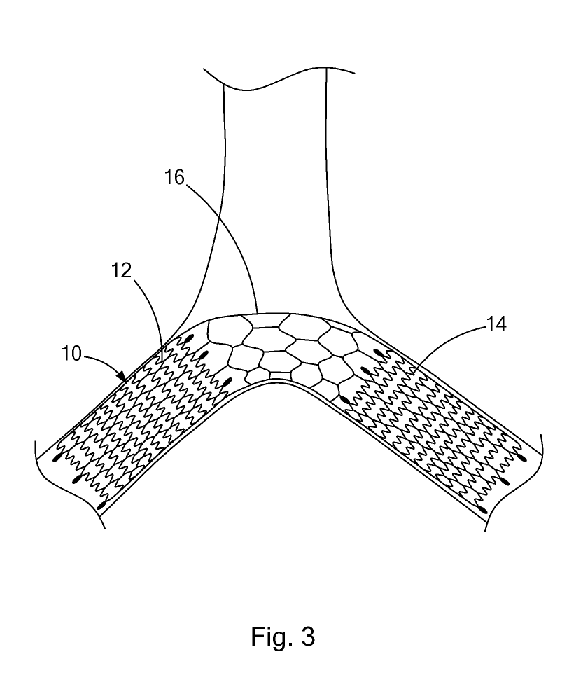

As shown, a filter portion 16 is connected to the first and second portions, 12 and 14 respectively. In this embodiment, the filter portion comprises a wire 40 formed into a third zig-zag configuration 42. However, it is understood that the wire 40 may be formed in any other configuration without falling beyond the scope or spirit of the present invention. The third configuration 42 comprises a series of straight sections 43 and a plurality of bends 44 connected to the straight sections 43. Preferably, the straight sections 43 are joined by the bends 44 defining a filter cell size of the filter portion 16. The filter portion 16 is configured to be disposed at the arc of the iliac veins at the opening of the inferior vena cava (see FIG. 3). The first and second portions 12 and 14 are configured to be opposingly disposed in respective left and right iliac veins of the patient. In another embodiment, the filter cell size is substantially the same as the first cell size. In yet another embodiment, the filter cell size is greater than the first cell size.

It is to be noted that the device 10 is depressible into a smaller first configuration or shape (FIG. 2) for delivery and deployment to a body vessel of a patient. The straight sections are arranged side-by-side and closely adjacent one another for insertion into the body vessel. In this embodiment, the bends store stress held within the first shape or configuration. The device is then expandable into a second shape or configuration (FIG. 1) by the release of the stress stored in the bends. The straight sections of the first and second portions press against the wall of the iliac veins to maintain the walls open while the filter portion filters thrombi therefrom.

As shown, the marker 27 may be disposed on one or more of the bends. In this embodiment, there is a plurality of markers where one of the markers is disposed on the first end and another marker is disposed on the second end of the first portion. The second portion 14 comprises a third end and a fourth end extending from the third end. One of the third end and the fourth end has a marker disposed thereon. As shown, the marker 37 is disposed on at least one of the bends. A plurality of markers is disposed on the third and the fourth end of the second portion. Thus, the filter portion is attached to the second end of the first portion and the third end of the second portion so that the filter portion is disposed between the first and second portions.

In this embodiment, the device 10 may be formed in any suitable manner, e.g., the device may be woven or laser cut as desired so long as the wires are configured to expand radially when the device is introduced into a body vessel.

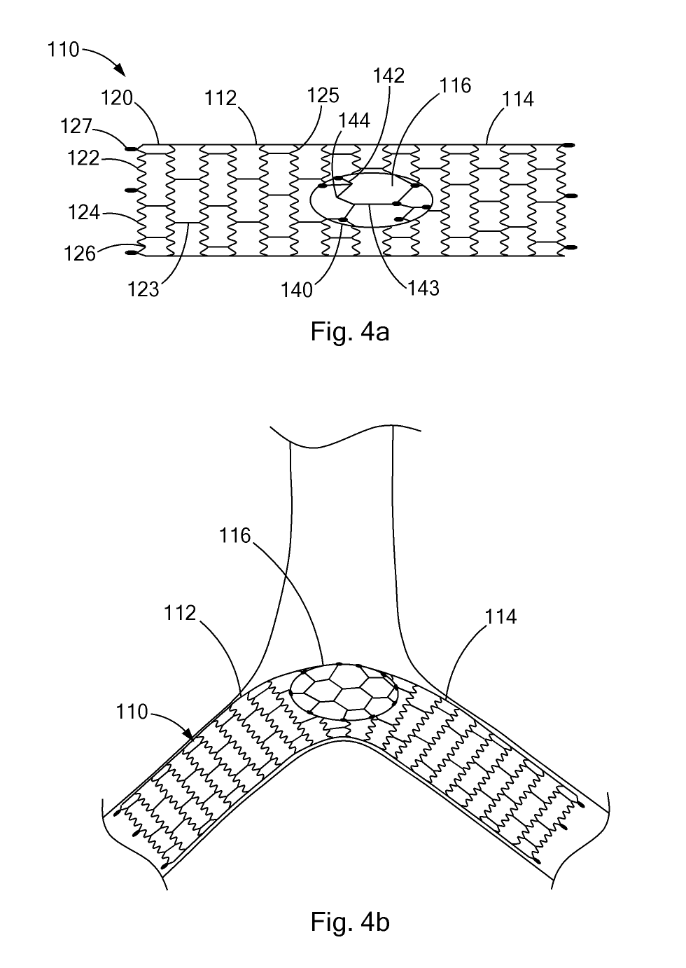

FIGS. 4a and 4b illustrate a filter device for capturing thrombi in a body vessel of a patient in accordance with another embodiment of the present invention. As shown, in FIG. 4a, the device 110 comprises a first portion 112 comprising a wire 120 formed into a first zig-zag configuration 122. The first configuration 122 comprises a series of straight sections 123 and a plurality of bends 126 connected to the straight sections 123.

In this embodiment, the straight sections 123 are joined by the bends 126 to define a first cell size of first portion 112. As shown, the first portion comprises a first end 124 and a second end 125 extending from the first end 124. Preferably, one of the first end 124 and second end 125 has a marker 127 disposed thereon.

As shown, a filter portion 116 is formed within the first portion 112. In this embodiment, the filter portion 116 comprises a wire 140 formed into a third filter configuration 142. The filter configuration 142 comprises a series of straight sections 143 and a plurality of bends 144 connected to the straight sections 143. Preferably, the straight sections 143 are joined by the bends 144 defining a filter cell size of the filter portion 116. The filter portion 116 is configured to be disposed at the arc of the iliac veins at the opening of the inferior vena cava (see FIG. 4b). The first portion 112 and the second portion 114 are configured to be opposingly disposed in respective left and right iliac veins of the patient.

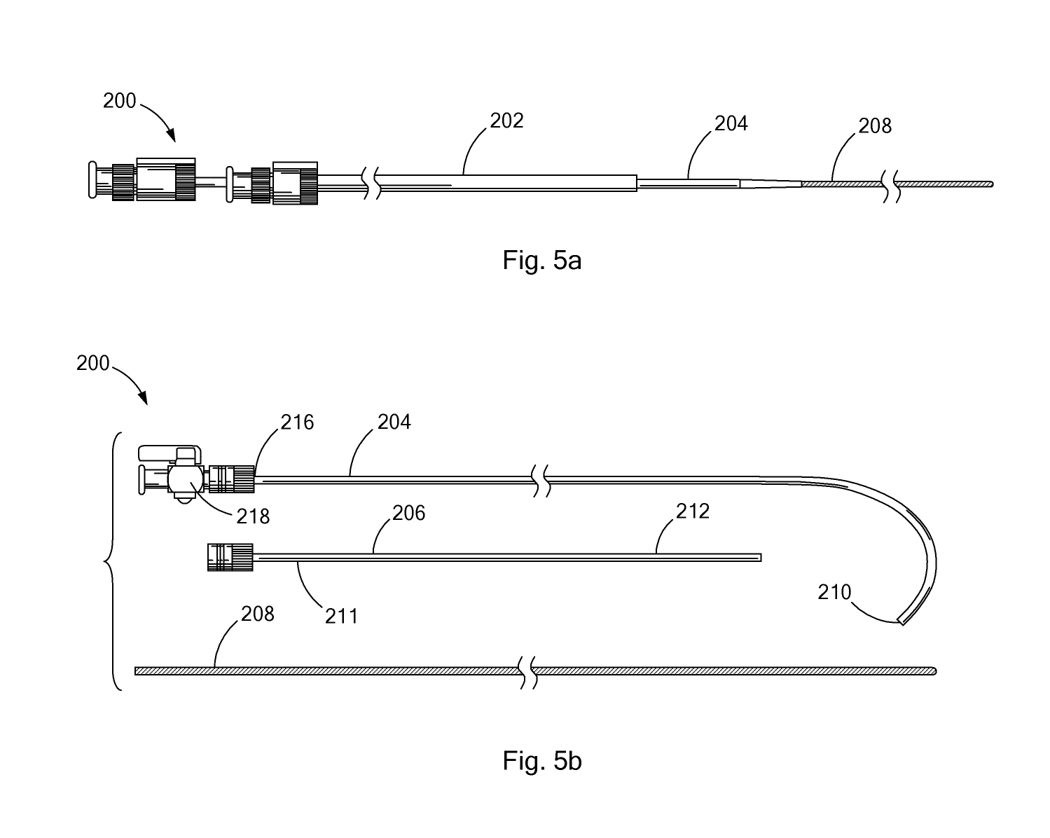

FIGS. 5a and 5b depict a delivery assembly 200 for introducing and retrieving the filter device for capturing thrombi in a body vessel in accordance with one embodiment of the present invention. As shown, the delivery assembly 200 includes a polytetrafluoroethylene (PTFE) introducer sheath 202 for percutaneously introducing an outer sheath 204 into a body vessel. Of course, any other suitable material for the introducer sheath 202 may be used without falling beyond the scope or spirit of the present invention. The introducer sheath 202 may have any suitable size, for example, between about 3-FR to 8-FR. The introducer sheath 202 serves to allow the outer sheath 204 and an inner member or catheter 206 to be percutaneously inserted to a desired location in the body vessel. The inner member may also include, for example, a stylet. The introducer sheath 202 receives the outer sheath 204 and provides stability to the outer sheath 204 at a desired location of the body vessel. For example, the introducer sheath 202 is held stationary within a common visceral artery, and adds stability to the outer sheath 204, as the outer sheath 204 is advanced through the introducer sheath 202 to a filter area in the vasculature. The outer sheath 204 has a body extending from a proximal end 216 to a distal end 210, the body being tubular and including a sheath lumen extending therethrough.

As shown, the assembly 200 may also include a wire guide 208 configured to be percutaneously inserted within the vasculature to guide the outer sheath 204 to the filter area. The wire guide 208 provides the outer sheath 204 with a path to follow as it is advanced within the body vessel. The size of the wire guide 208 is based on the inside diameter of the outer sheath 204 and the diameter of the target body vessel.

A needle may also be used. The needle may be used for percutaneously introducing the wire guide into the patient's body through an access site. A cutting device may also be used to expand the access site.

When the distal end 210 of the outer sheath 204 is at the desired location in the body vessel, the wire guide 208 is removed and the filter device 10, having a proximal segment contacting a distal portion 212 of the inner catheter 206, is inserted into the outer sheath 204. The inner catheter 206 is advanced through the outer sheath 204 for deployment of the filter device 10 through the distal end 210 to filter the body vessel. The catheter 206 extends from a proximal portion 211 to a distal portion 212 and is configured for axial movement relative to the outer sheath 204. In this example, the distal portion 212 is shown adjacent to the filter device 10 (similar to any of the filter devices described above). Thus, before deployment, the filter device 10 is coaxially disposed within the lumen of the outer sheath 204 and removably coupled to the distal portion 212 of the catheter 206, or in the alternative, the filter device 10 is merely pushed by, but not coupled to, the distal portion 212 of the catheter 206.

The outer sheath 204 further has a proximal end 216 and a hub 218 to receive the inner catheter 206 and filter device 10 to be advanced therethrough. The size of the outer sheath 204 is based on the size of the body vessel in which it percutaneously inserts, and the size of the filter device 10.

In this embodiment, the filter device 10 and inner catheter 206 are coaxially advanced through the outer sheath 204, following removal of the wire guide 208, in order to position the filter device 10 to filter the body vessel. The filter device 10 is guided through the outer sheath 204 by the inner catheter 206, preferably from the hub 218, and exits from the distal end 210 of the outer sheath 204 at a location within the vasculature where occlusion is desired. Thus, the filter device 10 is deployable through the distal end 210 of the outer sheath 204 by means of axial relative movement of the catheter 206. In order to more easily deploy the filter device 10 into the body vessel, the filter device 10 may have a lubricious coating, such as silicone or a hydrophilic polymer, e.g. AQ® Hydrophilic Coating as known in the art.

Likewise, this embodiment may also retrieve the filter device 10 by positioning the distal end 210 of the outer sheath 204 adjacent the deployed device 10 in the vasculature. The inner catheter 206 is advanced through the outer sheath 204 until the distal portion 212 protrudes from the distal end 210 of the outer sheath 204. The distal portion 212 is coupled to a proximal end of the filter device 10, after which the inner catheter 206 is retracted proximally, drawing the filter device 10 into the outer sheath 204.

It is understood that the assembly described above is merely one example of an assembly that may be used to deploy the filter device in a body vessel. Of course, other apparatus, assemblies and systems may be used to deploy any embodiment of the filter device without falling beyond the scope or spirit of the present invention.

As a person skilled in the art will readily appreciate, the above description is meant as an illustration of implementation of the principles this invention. This description is not intended to limit the scope or application of this invention in that the invention is susceptible to modification, variation and change, without departing from the spirit of this invention, as defined in the following claims.

1. A filter device for capturing thrombi in an iliac vein in a body vessel of a patient, the device comprising:
a first portion comprising a wire formed into a first zig-zag configuration, the first configuration comprising a series of straight sections and a plurality of bends connected to the straight sections, the straight sections being joined by the bends defining a first cell size of the first portion;
a second portion comprising the wire formed into a second zig-zag configuration, the second configuration comprising a series of straight sections and a plurality of bends connected to the straight sections, the straight sections being joined by the bends defining a second cell size of the second portion; and
a filter portion connected to an end of the first and second portions and being defined by a marker disposed on the end of the first and second portions, the filter portion comprising the wire formed into a third closed zig-zag configuration, the third configuration comprising a series of straight sections and a plurality of bends connected to the straight sections, the straight sections being joined by the bends defining a filter cell size of the filter portion, the filter cell size being greater than the first cell size, the filter portion being configured to be disposed at the arch of the iliac veins at the opening of the inferior vena cava, the first and second portions configured to be opposingly disposed in respective left and right iliac veins of the patient;
the device being depressible into a smaller first shape, the straight sections being arranged side-by-side and closely adjacent one another for insertion into the body vessel and the bends storing stress therein in the first shape, the device being expandable into a second shape by the release of the stress stored in the bends,
wherein the straight sections of the first and second portions press against the walls of the iliac veins to maintain the walls open while the filter portion filters thrombi there from.

2. The device of claim 1 wherein the first portion comprises a first end and a second end extending from the first end, one of the first end and the second end having the marker disposed thereon.

3. The device of claim 2 wherein the marker is disposed on one of the bends.

4. The device of claim 2 wherein the marker is a plurality of markers, one of the markers being disposed on the first end and another marker being disposed on the second end.

5. The device of claim 1 wherein the second portion comprises a first end and a second end extending from the first end, one of the first end and the second end having a marker disposed thereon.

6. The device of claim 5 wherein the marker is disposed on one of the bends.

7. The device of claim 5 wherein the marker is a plurality of markers, one of the markers being disposed on the first end and another marker being disposed on the second end.

8. The device of claim 1 wherein the first portion comprises a first end and a second end extending from the first end and wherein the second portion comprises a third end and a fourth end extending from the third end, the filter portion being attached to the second end of the first portion and the third end of the second portion so that the filter portion is disposed between the first and second portions.

9. The device of claim 1 wherein the first zig-zag configuration is one of an open configuration and a closed configuration.

10. The device of claim 1 wherein the second zig-zag configuration is one of an open configuration and a closed configuration.

11. The device of claim 1 wherein the first cell size is substantially the same as the second cell size.

12. A delivery assembly for placing a filter device for capturing thrombi in an iliac vein in a body vessel of a patient, the assembly comprising:
an outer sheath having a body extending from a proximal part to a distal part, the body being tubular and forming a sheath lumen extending there through;
an inner member extending from a proximal portion to a distal portion, the inner member being disposed within the sheath lumen and configured for axial movement relative to the outer sheath;
the filter device being coaxially disposed within the sheath lumen and disposed adjacent the distal portion of the inner member, the filter device being deployable through the distal part of the outer sheath by means of the relative axial movement of the inner member, the filter device comprising:
a first portion comprising a wire formed into a first zig-zag configuration, the first configuration comprising a series of straight sections and a plurality of bends connected to the straight sections, the straight sections being joined by the bends defining a first cell size of the first portion;
a second portion comprising the wire formed into a second zig-zag configuration, the second configuration comprising a series of straight sections and a plurality of bends connected to the straight sections, the straight sections being joined by the bends defining a second cell size of the second portion; and
a filter portion connected to an end of the first and second portions and being defined by a marker disposed on the end of the first and second portions, the filter portion comprising the wire formed into a third closed zig-zag configuration, the third configuration comprising a series of straight sections and a plurality of bends connected to the straight sections, the straight sections being joined by the bends defining a filter cell size of the filter portion, the filter cell size being greater than the first cell size, the filter portion being configured to be disposed at the arch of the iliac veins at the opening of the inferior vena cava, the first and second portions configured to be opposingly disposed in respective left and right iliac veins of the patient;
the device being depressible into a smaller first shape, the straight sections being arranged side-by-side and closely adjacent one another for insertion into the body vessel and the bends storing stress therein in the first shape, the device being expandable into a second shape by the release of the stress stored in the bends,
wherein the straight sections of the first and second portions press against the walls of the iliac veins to maintain the walls open while the filter portion filters thrombi there from.

13. The assembly of claim 12 wherein the first portion comprises a first end and a second end extending from the first end, one of the first end and the second end having the marker disposed thereon.

14. The assembly of claim 13 wherein the marker is disposed on one of the bends.

15. The assembly of claim 13 wherein the marker is a plurality of markers, one of the markers being disposed on the first end and another marker being disposed on the second end.

16. The assembly of claim 12 wherein the second portion comprises a first end and a second end extending from the first end, one of the first end and the second end having a marker disposed thereon.

17. The assembly of claim 16 wherein the marker is disposed on one of the bends.

18. The assembly of claim 16 wherein the marker is a plurality of markers, one of the markers being disposed on the first end and another marker being disposed on the second end.

19. The assembly of claim 12 wherein the first portion comprises a first end and a second end extending from the first end and wherein the second portion comprises a third end and a fourth end extending from the third end, the filter portion being attached to the second end of the first portion and the third end of the second portion so that the filter portion is disposed between the first and second portions.

20. The device of claim 12 wherein the first cell size is substantially the same as the second cell size.
